(19) United States
(12) Patent Application Publication
(10) Pub. No.: US 2018/0259561 A1
(43) Pub. Date: Sep. 13, 2018

(54) WEARABLE CURRENT SENSOR

(71) Applicant: ELECTRONICS AND TELECOMMUNICATIONS RESEARCH INSTITUTE, Daejeon (KR)

(72) Inventors: Chan Woo PARK, Daejeon (KR); Jae Bon KOO, Daejeon (KR)

(73) Assignee: ELECTRONICS AND TELECOMMUNICATIONS RESEARCH INSTITUTE, Daejeon (KR)

(21) Appl. No.: 15/721,823

(22) Filed: Sep. 30, 2017

(30) Foreign Application Priority Data
Mar. 7, 2017 (KR) 10-2017-0029142

(51) Int. Cl. G01R 29/08; G08B 21/18; G01R 33/02; G01R 33/00; G01R 33/04; G01R 15/12; G01R 15/18; A61B 5/11; A61B 5/00

(52) U.S. Cl. CPC G01R 29/0878; G08B 21/182; G01R 33/0206; G01R 33/0005; A61B 5/6804; G01R 15/12; G01R 15/181; A61B 5/1126; G01R 33/04

(57) ABSTRACT

A wearable current sensor according to embodiments of the inventive concepts includes a core, a coil wound on the core to surround the core, and a measurement part measuring an induced current induced in the coil. The coil includes a fiber having elasticity and a liquid metal in the fiber.

### CROSS-REFERENCE TO RELATED APPLICATION

This U.S. non-provisional patent application claims priority under 35 U.S.C. § 119 to Korean Patent Application No. 10-2017-0029142, filed on Mar. 7, 2017, in the Korean Intellectual Property Office, the disclosure of which is hereby incorporated by reference in its entirety.

### BACKGROUND

Embodiments of the inventive concepts relate to a current sensor and, more particularly, to a wearable current sensor.

Workers installing and checking electrical equipment may be exposed to a risk of electric shock during work. In particular, when a worker gets a low-voltage electric shock, muscles of the shocked worker may be contracted, and thus the shocked worker may not escape from the situation himself. In addition, a rescuer may be exposed to a risk of secondary electric shock in rescuing the shocked worker. Therefore, new techniques capable of quickly detecting and notifying of electric shock are needed to prevent personal injury caused by an electric shock accident. In other words, there is a need for a sensor which is attached to a body or clothes of a worker to measure a current flowing through the body in real time without interfering with work and which quickly transmits information on a situation or warns of electric shock when the electric shock occurs.

### SUMMARY

Embodiments of the inventive concepts may provide a wearable current sensor having elasticity.

In an aspect, a wearable current sensor may include a core, a coil wound on the core to surround the core, and a measurement part measuring an induced current induced in the coil. The coil may include a fiber having elasticity, and a liquid metal in the fiber.

In some embodiments, the liquid metal may include gallium (Ga).

In some embodiments, the core may include a soft magnetic body having elasticity.

In some embodiments, the core may include a composite composed of soft magnetic powder and an elastic polymer.

In some embodiments, the core may further include soft magnetic bulks, and the composite may be provided between the soft magnetic bulks.

In some embodiments, the core may include a tube having elasticity, and soft magnetic powder in the tube.

In some embodiments, the core may further include soft magnetic bulks disposed in the tube.

In some embodiments, the wearable current sensor may further include a warning generator comparing the induced current measured from the measurement part with a predetermined critical current to generate a warning when the induced current exceeds the critical current.

In some embodiments, the core may have a ring shape.

In an aspect, a wearable current sensor may include a core having a ring shape and surrounding a measurement target body, a coil wound on the core, a measurement part measuring a value of an induced current induced in the coil by a current applied to the measurement target body, and a warning generator generating a warning when the value of the induced current measured from the measurement part exceeds a value of a predetermined critical current. The core may include a composite composed of a soft magnetic material and an elastic polymer.

In some embodiments, the soft magnetic material may include at least one of sendust (Fe—Si—Al), megaflux (Fe—Si), molybdenum permalloy powder (MPP), high flux (Ni—Fe), or ferrite powder, and the elastic polymer may include at least one of polydimethylsiloxane (PDMS), polyurethane, silicone rubber, or styrene butadiene rubber (SBR).

In some embodiments, the core may further include a soft magnetic bulk, and the soft magnetic bulk may include at least one of permalloy (a Ni—Fe-based alloy), sintered ferrite, or silicon-steel (a Fe—Si alloy).

In some embodiments, the coil may include a liquid metal.

### BRIEF DESCRIPTION OF THE DRAWINGS

The inventive concepts will become more apparent in view of the attached drawings and accompanying detailed description.

FIG. 1 is a schematic view illustrating measurement of an induced current by using an electromagnetic induction phenomenon.

FIG. 2 is a schematic view illustrating a wearable current sensor according to some embodiments of the inventive concepts.

FIG. 3 is a view illustrating the wearable current sensor of FIG. 2, which is attached to the human body.

FIG. 4A is a view illustrating a core according to some embodiments of the inventive concepts.

FIG. 4B is a view illustrating a composite of the core of FIG. 4A.

FIG. 4C is a view illustrating a core according to some embodiments of the inventive concepts.

FIG. 4D is a view illustrating a core according to some embodiments of the inventive concepts.

FIG. 4E is a view illustrating a core according to some embodiments of the inventive concepts.

FIG. 5 is a view illustrating a coil according to some embodiments of the inventive concepts.

### DETAILED DESCRIPTION OF THE EMBODIMENTS

The inventive concepts will now be described more fully hereinafter with reference to the accompanying drawings, in which exemplary embodiments of the inventive concepts are shown. The advantages and features of the inventive concepts and methods of achieving them will be apparent from the following exemplary embodiments that will be described in more detail with reference to the accompanying drawings. It should be noted, however, that the inventive concepts are not limited to the following exemplary embodiments, and may be implemented in various forms. Accordingly, the exemplary embodiments are provided only to disclose the inventive concepts and let those skilled in the art know the category of the inventive concepts. In the drawings, embodiments of the inventive concepts are not limited to the specific examples provided herein and are exaggerated for clarity.

The terminology used herein is for the purpose of describing particular embodiments only and is not intended to limit the invention. As used herein, the singular terms “a”, “an” and “the” are intended to include the plural forms as well, unless the context clearly indicates otherwise. As used herein, the term “and/or” includes any and all combinations of one or more of the associated listed items. It will be understood that when an element is referred to as being “connected” or “coupled” to another element, it may be directly connected or coupled to the other element or intervening elements may be present. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Moreover, exemplary embodiments are described herein with reference to cross-sectional illustrations and/or plane illustrations that are idealized exemplary illustrations. In the drawings, the thicknesses of layers and regions are exaggerated for clarity. Accordingly, variations from the shapes of the illustrations as a result, for example, of manufacturing techniques and/or tolerances, are to be expected. Thus, exemplary embodiments should not be construed as limited to the shapes of regions illustrated herein but are to include deviations in shapes that result, for example, from manufacturing. For example, an etching region illustrated as a rectangle will, typically, have rounded or curved features. Thus, the regions illustrated in the figures are schematic in nature and their shapes are not intended to illustrate the actual shape of a region of a device and are not intended to limit the scope of example embodiments.

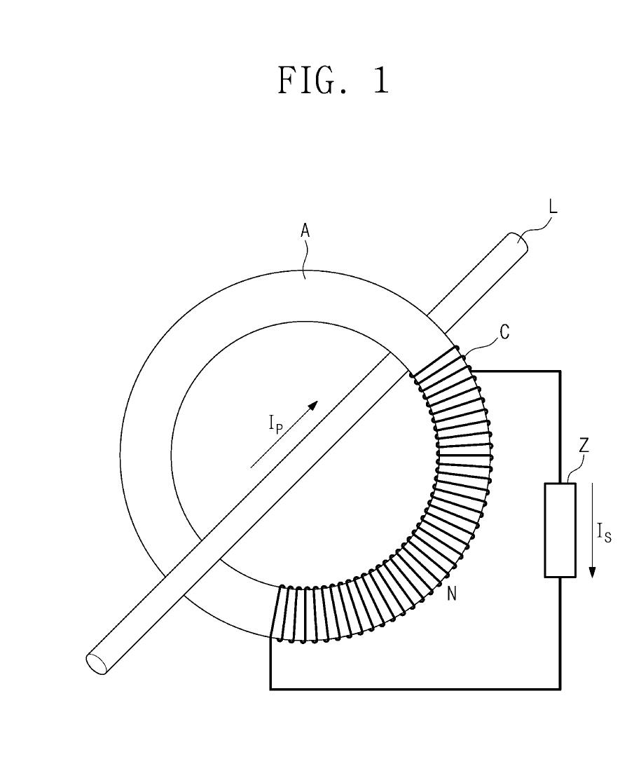

FIG. 1 is a schematic view illustrating measurement of an induced current I_{S} by using an electromagnetic induction phenomenon. A conducting wire L may be provided inside a core A having a ring shape, and a secondary coil C may be wound on the core A. Impedance Z may be provided to the secondary coil C. For example, the impedance Z may be an inner resistance of a current measuring device.

A magnetic field may be generated around the conducting wire L by a current I_{P} applied to the conducting wire L. The current I_{P} may be an alternating current. Hereinafter, the alternating current I_{P} will be described as an example of the current I_{P}. An intensity of the magnetic field generated around the conducting wire L may be amplified inside the core A surrounding the conducting wire L. The intensity of the magnetic field inside the core A may be varied as the alternating current I_{P} applied to the conducting wire L is varied. Thus, induced electromotive force for counteracting the variation in the magnetic field may be generated in the secondary coil C, and an induced current I_{S} may flow through the secondary coil C. Here, the alternating current I_{P} applied to the conducting wire L, the induced current I_{S} induced in the secondary coil C, and the number N of turns of the secondary coil C satisfy the following equation.

$${I}_{S} = \frac{{I}_{P}}{N} \qquad [ Equation ]$$

Thus, the alternating current I_{P} may be reversely calculated using the induced current I_{S} induced in the secondary coil C and the number N of turns of the secondary coil C.

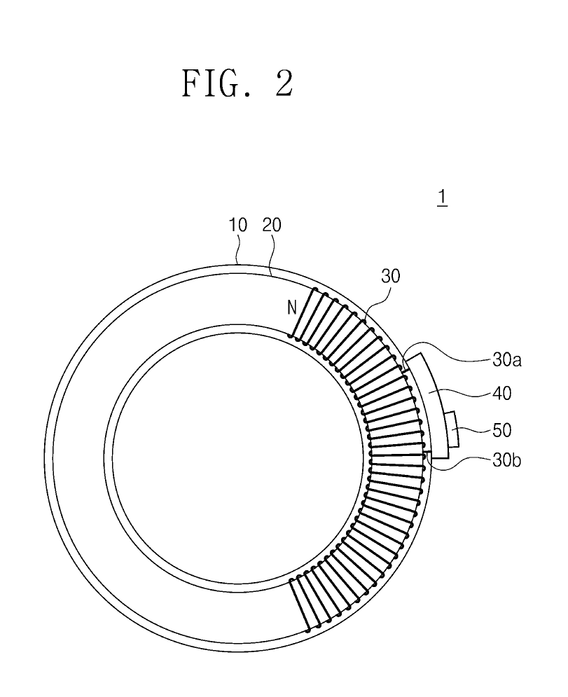

FIG. 2 is a schematic view illustrating a wearable current sensor 1 according to some embodiments of the inventive concepts. Referring to FIG. 2, a wearable current sensor 1 may include a body 10, a core 20, a coil 30, a measurement part 40, and a warning generator 50. The wearable current sensor 1 may have elasticity, unlike a general current tester. Since the wearable current sensor 1 has the elasticity, the wearable current sensor 1 may be easily attached to and detached from a human body or clothes of a worker.

The body 10 may be formed of an elastic material. The body 10 may be formed of an insulating material. In some embodiments, the body 10 may have a tube shape.

The core 20 may be provided in the body 10. The core 20 may have a ring shape. For example, the core 20 may have a circular ring shape. However, embodiments of the inventive concepts are not limited thereto. A measurement target body (e.g., a human body) may be provided inside the ring-shaped core 20, and the core 20 may surround the measurement target body. The core 20 may include a soft magnetic body having elasticity. A material and ingredients of the soft magnetic body of the core 20 will be described later.

The coil 30 may surround the core 20. For example, the coil 30 may be wound on the core 20. The coil 30 may surround at least a portion of the core 20, and both end portions 30a and 30b of the coil 30 may be connected to the measurement part 40. The number N of turns of the coil 30 may be adjusted according to needs of a worker. The coil 30 may include a material having elasticity. The material and ingredients of the coil 30 will be described later.

The measurement part 40 may be coupled to the body 10. In some embodiments, the measurement part 40 may be coupled to an outer surface of the body 10. The measurement part 40 may be electrically connected to the coil 30 to measure a value of an induced current which is induced in and flows through the coil 30.

The warning generator 50 may be coupled to the body 10. The warning generator 50 may be coupled to the outer surface of the body 10. In some embodiments, the warning generator 50 may be coupled to the measurement part 40. However, embodiments of the inventive concepts are not limited to the described arrangement of the warning generator 50 and the measurement part 40. The warning generator 50 may compare the value of the induced current, measured by the measurement part 40, with a value of a predetermined critical current and may generate a warning when the value of the induced current exceeds the value of the critical current. The warning generator 50 may transmit information on the measured induced current to an external device electrically connected thereto when the value of the induced current exceeds the value of the critical current. The warning may be provided in the form of an alarm, a buzzer, or an external transmission signal. For example, the value of the critical current may range from about 5 mA to about 100 mA.

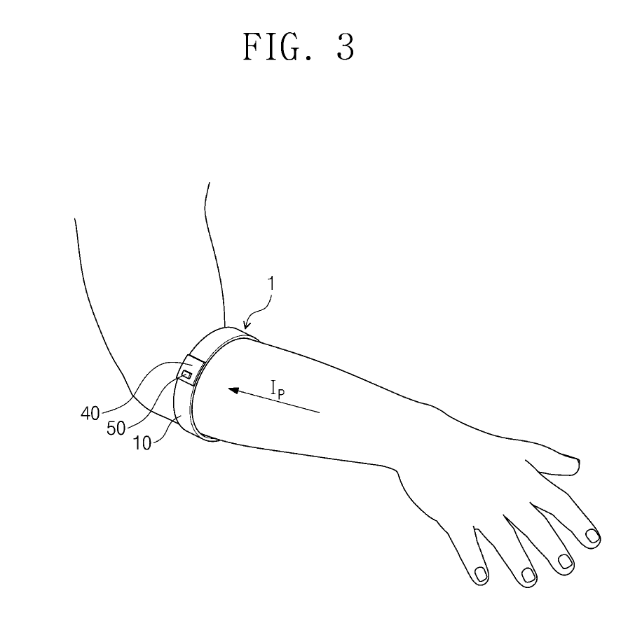

FIG. 3 is a view illustrating the wearable current sensor 1 of FIG. 2, which is attached to the human body. As described above, the measurement part 40 and the warning generator 50 may be coupled to the outer surface of the body 10. As described with reference to FIG. 1, the wearable current sensor 1 may calculate a value of an alternating current, flowing through the human body, by using the number N of turns of the coil 30 and the value of the induced current of the coil 30 measured by the measurement part 40. The wearable current sensor 1 may generate an alarm when the value of the induced current exceeds the value of the predetermined critical current. The wearable current sensor 1 may transmit information on the measured induced current to an external device electrically connected thereto when the value of the induced current exceeds the value of the critical current.

Since the wearable current sensor 1 has the elasticity, the wearable current sensor 1 may be easily attached to and/or detached from the human body or clothes and may reduce an influence on working activity. In some embodiments, a length of the wearable current sensor 1 may can be varied to a specific value ranging from about 110% to about 150% of an initial length. Here, the length of the wearable current sensor 1 may mean a circumferential length of the wearable current sensor 1. In addition, even though not shown in the drawings, the wearable current sensor 1 may further include a display part that shows the measured value of the induced current.

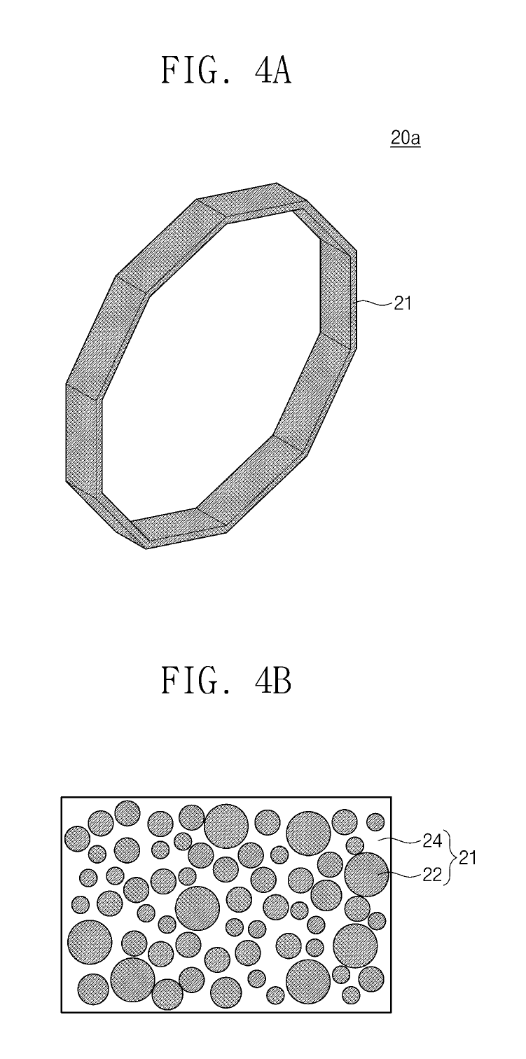

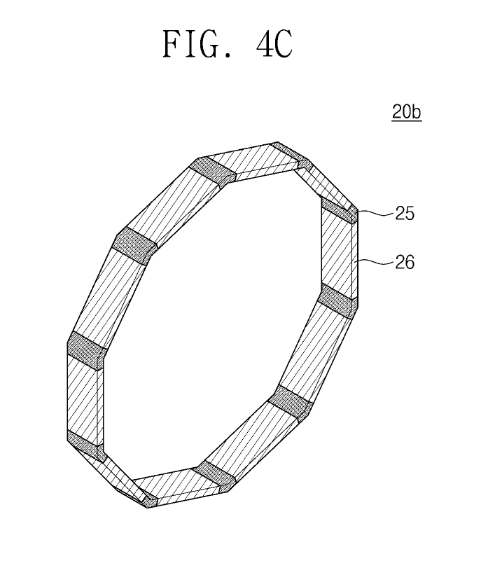

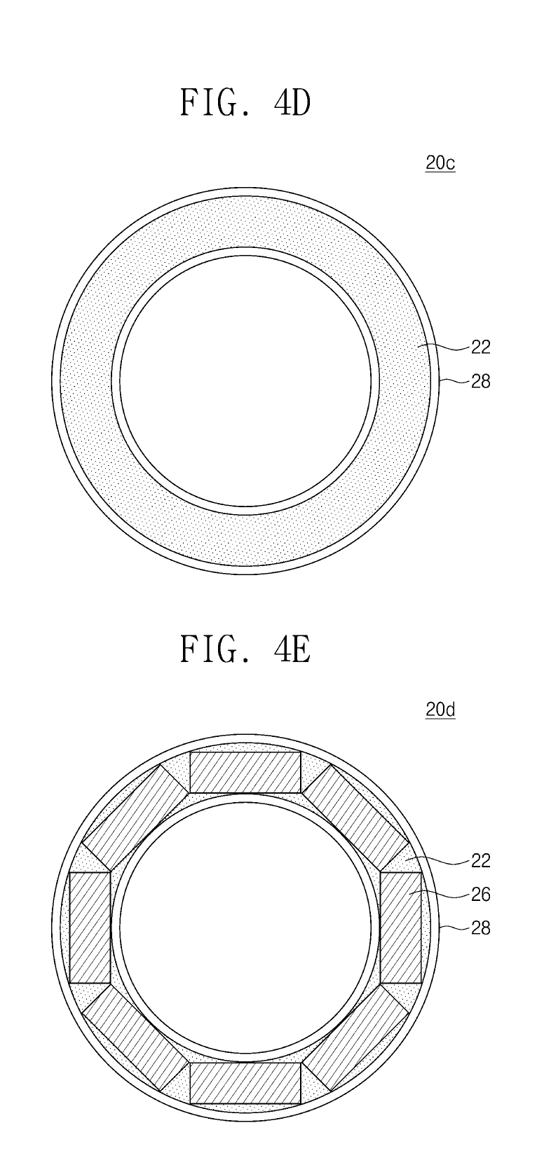

FIG. 4A is a view illustrating a core 20a according to some embodiments of the inventive concepts. FIG. 4B is a view illustrating a composite of the core 20a of FIG. 4A. The core 20a may have, but not limited to, a polygonal ring shape. The core 20a may be formed of a composite 21.

Referring to FIGS. 4A and 4B, the composite 21 may include soft magnetic powder 22 and an elastic polymer 24. The soft magnetic powder 22 may include at least one of sendust (Fe—Si—Al), megaflux (Fe—Si), molybdenum permalloy powder (MPP), high flux (Ni—Fe), or ferrite powder. In the present specification, the soft magnetic powder 22 may mean particles of which diameters are in a range of several nanometers (nm) to tens micrometers (μm). The elastic polymer 24 may include at least one of polydimethylsiloxane (PDMS), polyurethane, silicone rubber, or styrene butadiene rubber (SBR).

In some embodiments, the soft magnetic powder 22 may be mixed with the elastic polymer 24, and then, a hardener may be added to the mixture of the soft magnetic powder 22 and the elastic polymer 24 to form the composite 21. However, the ingredients and formation method of the composite 21 are not limited thereto.

FIG. 4C is a view illustrating a core 20b according to some embodiments of the inventive concepts. Hereinafter, the descriptions to the same features as in the core 20a of FIGS. 4A and 4B will be omitted or mentioned briefly for the purpose of ease and convenience in explanation.

The core 20b may have a polygonal ring shape, but may not be limited thereto. The core 20b may include bulks 26 and coupling portions 25. The bulks 26 may include a soft magnetic material. In some embodiments, the bulks 26 may include at least one of, but not limited to, permalloy (a Ni—Fe-based alloy), sintered ferrite, or silicon-steel (a Fe—Si alloy). Each of the bulks 26 may have a plate or rod shape and may mean a body having a length ranging from several millimeters (mm) to tens centimeters (cm). Each of the coupling portions 25 may be disposed between the bulks 26 to couple the bulks 26 adjacent thereto, and thus the bulks 26 may be coupled in one united body by the coupling portions 25. The coupling portions 25 may be formed of the composite described above with reference to FIGS. 4A and 4B.

FIG. 4D is a view illustrating a core 20c according to some embodiments of the inventive concepts. Hereinafter, the same elements as described in the core 20a of FIGS. 4A and 4B will be indicated by the same reference numerals, and the descriptions thereto will be omitted or mentioned briefly for the purpose of ease and convenience in explanation.

The core 20c may include a first fiber 28 and soft magnetic powder 22 provided in the first fiber 28. The first fiber 28 may have elasticity. For example, the first fiber 28 may be formed of an elastic material such as silicon or polyurethane. The soft magnetic powder 22 may be provided to fill an inner space of the first fiber 28. The soft magnetic powder 22 may include at least one of sendust (Fe—Si—Al), megaflux (Fe—Si), molybdenum permalloy powder (MPP), high flux (Ni—Fe), or ferrite powder.

FIG. 4E is a view illustrating a core 20d according to some embodiments of the inventive concepts. Hereinafter, the same elements as described in the core 20c of FIG. 4D will be indicated by the same reference numerals, and the descriptions thereto will be omitted or mentioned briefly for the purpose of ease and convenience in explanation.

The core 20d may further include bulks 26 provided in the first fiber 28. The bulks 26 may include a soft magnetic material. Each of the bulks 26 may have a plate or rod shape and may mean a body having a length ranging from several millimeters (mm) to tens centimeters (cm). The bulks 26 may be disposed in the first fiber 28, and the soft magnetic powder 22 may fill spaces between the bulks 26 in the first fiber 28.

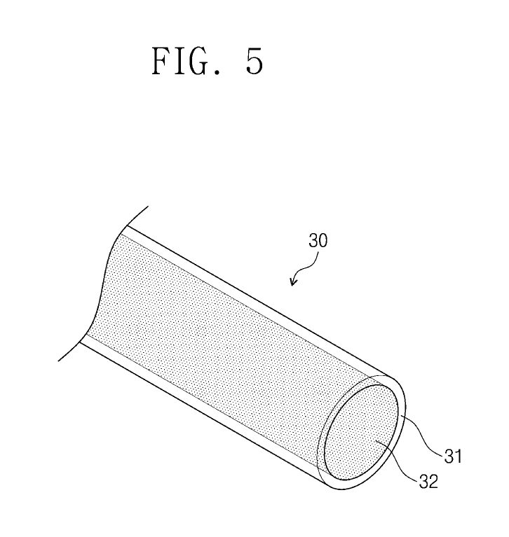

FIG. 5 is a view illustrating a coil 30 according to some embodiments of the inventive concepts. The coil 30 may include a second fiber 31 and a liquid metal 32. The second fiber 31 may have elasticity. For example, the second fiber 31 may be formed of an elastic material such as silicon or polyurethane. An inner space of the second fiber 31 may be filled with the liquid metal 32. For example, the liquid metal 32 may include gallium (Ga). Even though a shape or a volume of the second fiber 31 is changed by the elasticity of the second fiber 31, the coil 30 may have high electrical conductivity due to the liquid metal 32 provided in the second fiber 31.

According to some embodiments of the inventive concepts, the wearable current sensor with excellent elasticity may be provided. In addition, the wearable current sensors according to embodiments of the inventive concepts may be easily attached to and/or detached from the human body and/or clothes of a worker and may monitor a magnitude of the alternating current flowing through the body of the worker in real time.

The cores 20a to 20d according to the aforementioned embodiments are examples of the core 20 including the soft magnetic body having the elasticity. However, the material, ingredients or any combination thereof in the core 20 including the soft magnetic body having the elasticity are not limited thereto. In addition, according to some embodiments of the inventive concepts, both the core and the coil may be formed of elastic materials, and thus the wearable current sensors having the elasticity may be provided or realized.

According to some embodiments of the inventive concepts, the wearable current sensor with excellent elasticity may be realized. In addition, the wearable current sensors according to embodiments may be easily attached to and/or detached from the human body and/or clothes of a worker and may monitor a magnitude of the alternating current flowing through the body of the worker in real time. Furthermore, the wearable current sensors may generate a warning (e.g., an alarm) when necessary.

While the inventive concepts have been described with reference to example embodiments, it will be apparent to those skilled in the art that various changes and modifications may be made without departing from the spirits and scopes of the inventive concepts. Therefore, it should be understood that the above embodiments are not limiting, but illustrative. Thus, the scopes of the inventive concepts are to be determined by the broadest permissible interpretation of the following claims and their equivalents, and shall not be restricted or limited by the foregoing description.

1. A wearable current sensor comprising:
a core;
a coil wound on the core to surround the core;
a body housing the core and the coil;
a measurement part measuring an induced current induced in the coil, the measurement part coupled to an outer surface of the body; and
a warning generator configured to generate a warning based on the measurement part measuring a predetermined induced current in the coil, the warning generator coupled to the body,
wherein the coil comprises:
a fiber having elasticity; and
a liquid metal in the fiber.

2. The wearable current sensor of claim 1, wherein the liquid metal includes gallium (Ga).

3. The wearable current sensor of claim 1, wherein the core comprises a soft magnetic body having elasticity, such that a circumferential length of the core is variable.

4. The wearable current sensor of claim 3, wherein the core comprises a composite composed of soft magnetic powder and an elastic polymer.

5. The wearable current sensor of claim 4, wherein the core further comprises soft magnetic bulks, and
wherein the composite is provided between the soft magnetic bulks.

6. The wearable current sensor of claim 3, wherein the core comprises:
a tube having elasticity; and
soft magnetic powder in the tube.

7. The wearable current sensor of claim 6, wherein the core further comprises: soft magnetic bulks disposed in the tube.

8. The wearable current sensor of claim 1, wherein the warning generator compares the induced current measured from the measurement part with a predetermined critical current to generate a warning when the induced current exceeds the critical current.

9. The wearable current sensor of claim 1, wherein the core has a ring shape.

10. A wearable current sensor comprising:
a core having a ring shape and surrounding a measurement target body;
a coil wound on the core;
a body housing the core and the coil;
a measurement part measuring a value of an induced current induced in the coil by a current applied to the measurement target body, the measurement part coupled to an outer surface of the body; and
a warning generator generating a warning when the value of the induced current measured from the measurement part exceeds a value of a predetermined critical current, the warning generator coupled to the body,
wherein the core comprises a composite composed of a soft magnetic material and an elastic polymer, and
wherein the core and body are elastically variable in a circumferential length direction.

11. The wearable current sensor of claim 10, wherein the soft magnetic material includes at least one of sendust (Fe—Si—Al), megaflux (Fe—Si), molybdenum permalloy powder (MPP), high flux (Ni—Fe), or ferrite powder, and
wherein the elastic polymer includes at least one of polydimethylsiloxane (PDMS), polyurethane, silicone rubber, or styrene butadiene rubber (SBR).

12. The wearable current sensor of claim 10, wherein the core further comprises a soft magnetic bulk, and
wherein the soft magnetic bulk includes at least one of permalloy (a Ni—Fe-based alloy), sintered ferrite, or silicon-steel (a Fe—Si alloy).

13. The wearable current sensor of claim 10, wherein the coil includes a liquid metal.
